(19) United States
(12) Patent Application Publication
(10) Pub. No.: US 2022/0184244 A1
(43) Pub. Date: Jun. 16, 2022

(54) THERMAL DISINFECTION DEVICE AND DISINFECTION METHOD USING SAME

(71) Applicant: SPECTRONIX INC., Gatineau (CA)

(72) Inventor: Patrick Assouad, Ottawa (CA)

(21) Appl. No.: 17/553,154

(22) Filed: Dec. 16, 2021

Related U.S. Application Data
(60) Provisional application No. 63/126,476, filed on Dec. 16, 2020.

(51) Int. Cl. A61L 2/06; A61L 2/24

(52) U.S. Cl. CPC A61L 2/06; A61L 2/24; A61L 2202/15; A61L 2202/14; A61L 2202/122

(57) ABSTRACT

Described are various embodiments of a thermal disinfection device and disinfection method using same. In one example, the thermal disinfection device is for disinfecting an article susceptible to thermal damage, and comprises a receptacle dimensioned to hold the article; a heater; a plurality of thermal probes dimensioned and independently displaceable to measure a respective temperature; and a controller operable to receive respective temperature readings and control the heater so to maintain a temperature reading at or above a preset temperature for a corresponding preset time period suitable to disinfect the article without inflicting damage to the article.

### CROSS REFERENCE TO RELATED APPLICATIONS

This application claims priority to U.S. Provisional Application No. 63/126,476 filed Dec. 16, 2020, the entire disclosure of which is incorporated herein by reference.

### FIELD OF THE DISCLOSURE

The present disclosure relates to disinfection systems, and, in particular, to a thermal disinfection device, system, and disinfection method using same.

### BACKGROUND

Established disinfection methods are generally based on chemicals (ozone, peroxide, etc.), radiation (UV, etc.), and heat. Chemicals and radiation require proper training, infrastructure support, labor, and safety protocols to reduce exposure. Excess heat may also degrade materials. In addition, UV light is effective in a line of sight geometry, making it very difficult to treat complex shapes. Most systems have been designed for medical and industrial applications (such as food) that do not have requirements for mobility and ease of use, or limitations to infrastructure (power, water, supplies, etc.).

Other systems exist like disinfectors used in clinical and dental practices such as the Hygojet™ from Dürr Dental, where items are washed and rinsed with hot water, but these systems typically require special detergents and are not meant to be mobile. They use hot water with limited control, which is not optimal as the even distribution of temperature over the surface of the item is not assured. Indeed, some items have high heat capacity that affect surrounding temperatures and complex geometries that impede the circulation of water.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a mobile disinfection system and method using same that overcome some of the drawbacks of known techniques, or at least, provides a useful alternative thereto.

In accordance with one aspect, there is provided a thermal disinfection device for disinfecting an article susceptible to thermal damage, the device comprising: a receptacle dimensioned to hold the article within a heating fluid; a heater; a plurality of thermal probes dimensioned and independently displaceable to be located at various locations on, in or within the article when in said receptacle, to measure a respective temperature of the heating fluid at each of said locations; and a controller operable to receive as input respective temperature readings from said thermal probes from said various locations, and control said heater so to maintain said temperature reading at or above a preset temperature for a corresponding preset time period suitable to disinfect the article without inflicting damage to the article.

In one embodiment, of said thermal probes comprises a wireless thermal probe.

In one embodiment, the device further comprises a wireless receiver operatively coupled to the controller for receiving said respective temperature readings.

In one embodiment, the fluid comprises water, and wherein the device further comprises a sealable submersible compartment in which to dispose the article, wherein said compartment comprises a wireless receiver disposed thereon to wirelessly interface with each said wireless thermal probe from within said compartment.

In one embodiment, the fluid comprises water, and wherein the device further comprises a sealable submersible compartment in which to dispose the article, wherein said compartment comprises a wiring interface to wired connections with each of said thermal probes from within said compartment, and an external wired or wireless connection via said wiring interface to said controller.

In one embodiment, the device further comprises an ambient thermal probe to measure an ambient temperature around the article, wherein said controller is further operable to limit said ambient temperature exceeding a preset thermal damage threshold.

In one embodiment, the device further comprises a mixing device for enhancing fluid circulation within said receptacle.

In one embodiment, the fluid comprises water and wherein said mixing device comprises an induction-controlled rotating linear magnet.

In one embodiment, the fluid comprises air and wherein said mixing device comprises a fan.

In one embodiment, the preset temperature is about 70 C and said preset time period is about 20 minutes.

In one embodiment, the controller further comprises an interface to adjust said preset temperature, and wherein said controller is operable to automatically adjust said corresponding preset time period according to said adjusted preset temperature.

In one embodiment, the controller further comprises a user interface providing for selection from a plurality of preset disinfection cycles preset for a corresponding set of designated articles having a preset disinfection temperature and time associated therewith consistent with a previously tested disinfection level.

In one embodiment, the probes are dimensioned so to be disposable within a recess or cavity of the article.

In one embodiment, the receptacle is hermetically sealed in operation for air-based thermal disinfection.

In one embodiment, at least some of said receptacle is manufactured of a collapsible material to allow for collapse and deployment of the device.

In one embodiment, the collapsible material comprise a water impermeable fabric.

In one embodiment, the fluid is selectable between water or air.

In accordance with another aspect, there is provided a method for disinfecting an article susceptible to thermal damage, the method comprising: locating a plurality of thermal probes on, in or within the article in accordance with a preset probe layout defined for the article; containing the article within a heating fluid; heating the heating fluid so to maintain respective temperature readings from said thermal probes at or above a preset disinfection temperature for a corresponding preset time period suitable to disinfect the article without inflicting thermal damage to the article.

In one embodiment, the method further comprises, prior to said heating, selecting a preset disinfection cycle from a plurality of selectable preset disinfection cycles defined for different articles and/or disinfection levels, wherein said heating is automatically implemented as a function of said selecting.

In accordance with another aspect, here is provided a thermal disinfection device for disinfecting an article having multiple recesses defined therein and susceptible to thermal damage, the device comprising: a receptacle dimensioned to hold the article submerged in water; a heater; a plurality of thermal probes dimensioned and independently displaceable to be located within the recesses when the article is submerged in water in said receptacle, to measure a respective water temperature within each of the recesses; and a controller operable to receive as input respective temperature readings from said thermal probes from within the recesses, and control said heater so to maintain said temperature reading at or above a preset temperature for a corresponding preset time period suitable to disinfect the article without inflicting damage to the article.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1A and FIG. 1B are schematic diagrams of a thermal disinfection device, in accordance with one embodiment;

FIG. 2A and FIG. 2B are schematic diagrams of a wireless thermal probe and sealable submersible compartment, in accordance with one embodiment;

FIG. 3 is a schematic diagram illustrating an exemplary wiring interface for a sealable submersible compartment, in accordance with one embodiment;

FIG. 4 is a schematic diagram of a multiplicity of ambient thermal probes, in accordance with one embodiment;

FIG. 5 is a schematic diagram of a mixing device comprising a linear magnet, in accordance with one embodiment;

FIG. 6 is a process flow diagram illustrating a thermal disinfection method, in accordance with one embodiment;

FIG. 7A is a top perspective view of a water-based thermal disinfection device, whereas FIG. 7B and FIG. 7C are top and bottom perspective views of an air-based thermal disinfection device and lid therefor, in accordance with respective exemplary embodiments;

FIG. 8A, FIG. 8B and FIG. 8C are perspective views of a deployable/ collapsible thermal disinfection device shown in a collapsed, exploded and deployed configuration, in accordance with one exemplary embodiment;

FIG. 9A, FIG. 9B, FIG. 9C and FIG. 9D are perspective views of a deployable/collapsible thermal disinfection device, wherein FIGS. 9A and 9B are top and bottom perspective views of the device in a collapsed configuration, FIG. 9C is a top perspective view of the device in a partially deployed state showing a deployment thereof, and FIG. 9D is a top perspective view of the device in a fully deployed state, in accordance with another exemplary embodiment;

FIG. 10A, FIG. 10B, FIG. 10C and FIG. 10D are perspective views of a deployable/collapsible thermal disinfection device, wherein FIG. 10A is a top perspective view of the device in a collapsed configuration, FIG. 10B is a top perspective view of the device in a partially deployed state, and FIGS. 10C and 10D are top and side perspective views of the device in a fully deployed state in both closed and open configurations, respectively, in accordance with another exemplary embodiment;

FIG. 11A, FIG. 11B and FIG. 11C are top perspective views of a deployable/collapsible thermal disinfection device, wherein FIGS. 11A and 11B are top perspective views of the device in a deployed state in both open and closed configurations, respectively, whereas FIG. 11C is an exploded view of an assembly of the device, in accordance with another exemplary embodiment; and

FIG. 12A, FIG. 12B and FIG. 12C are respective screenshots of a disinfection cycle selection and control interface for the device of FIG. 12A, in accordance with one embodiment.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

The systems and methods described herein provide, in accordance with different embodiments, different examples in which an article that is generally susceptible to thermal damage may be disinfected by a thermal disinfection device to at least 5 log10 cycles of disinfection (99.999%), which is equivalent to effective chemical sanitization. The main benefit of the device described below, in accordance with different embodiments, is the complete lack of exposure to chemicals for both the user and the equipment.

Being able to precisely control the temperature and time to which the material is exposed offers the opportunity to process sensitive equipment and fabrics to an effective 6 log10 level without causing damage. This may include articles sensitive to ultraviolet (UV) radiation and chemicals. The device and method described below only uses water at low enough temperatures so as to minimize or nullify any damage or increased wear on the item or article being disinfected. Controlled wet heat has the distinctive advantage of allowing an exact preset temperature and disinfection level depending on materials.

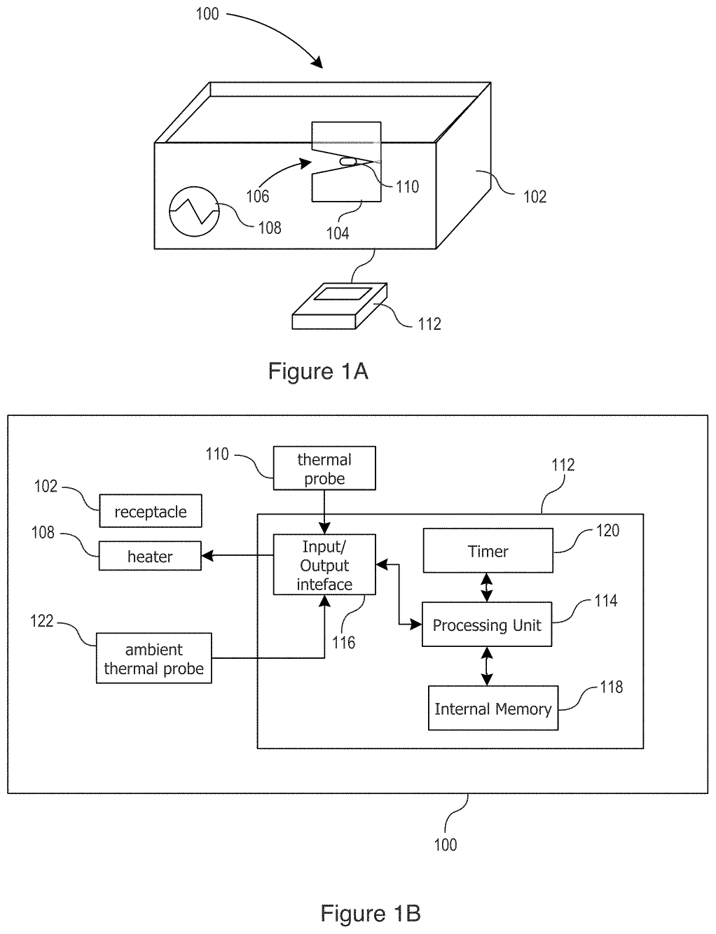

With reference to FIGS. 1A and 1B, and in accordance with one exemplary embodiment, a thermal disinfection device for disinfecting an article, generally referred to using the numeral 100, will now be described. Device 100 operates via the use of water to generate a wet heat that achieves levels of disinfection at comparatively lower temperatures then was is usually the case with steam, for example.

Thus, in some embodiments, device 100 comprises a receptacle, chamber or container 102 filled with water and dimensioned for receiving or holding an item or article 104 to be disinfected submerged therein. In some embodiments, receptacle 102 may be small enough so as to be mobile and versatile, so as to be used in almost any environment with a quick turnaround time and being readily transportable by a single person with minimal effort.

Generally, article 104 may have a complex shape and comprise hard to reach areas, e.g. generally referred to herein as recesses which may include, but are not limited to, grooves, folds (e.g. fabrics), creases, tubing, cavities, apertures, etc., such as exemplary recess 106 or similar. Examples of articles may include, without limitation, wearable items, apparel, equipment and/or mechanical or electronic devices, for example. Generally, device 100 may be used with any items comprising any material that can withstand getting moist for a short duration at a temperature above a minimum of 50° C. Alternatively, items that preferably are not to be put in contact with water, can be treated with embodiments of the device described herein by using a suitable sealable compartment, as described in greater detail below.

Device 100 further comprises a heater 108 which may include for example one or more heating elements operable to heat the water contained within receptacle 102. These may be located at different locations within receptacle 102.

In addition, at least one thermal probe 110, dimensioned and displaceable to be located within recess 106 when article 104 is submerged in water in receptacle 102, is provided to measure a water temperature within recess 106. Thermal probe 110 may take the form of a thermocouple, although other temperature sensors may be used. Different shapes or dimensions may be envisioned for thermal probe 110. On the larger side, for example and without limitation, a cylindrically shaped probe may have dimensions close to 3 cm long and a radius of 0.75 cm, while smaller miniaturized probes may have a size closer to 5 mm by 5 mm. In some embodiments, thermal probe 110 may be operable to be releasably attached to article 104. Thus, by strategically placing thermal probe 110 on or inside article 104, a good indication of whether the desired disinfection is reached everywhere may be acquired. Different means for releasably attaching thermal probe 110 may be envisioned. This may include, without limitation, an adhesive, a tie-wrap or similar, or a magnet for articles comprising magnetic surfaces. In other embodiments, thermal probe 110 may have a substantially elongated shape and may be configured to bend along its length so as to more easily reach hard to access areas of article 104, such as recess 106.

Device 100 further comprises a controller 112, operationally linked to the heating element 108 and thermal probe 110. As illustrated in FIG. 1B, controller 112 generally comprises a digital processing unit 114, an input/output interface 116 and internal memory 118. In some embodiments, it may also comprise a hardware timing device or timer 120 for measuring heating time periods or durations, although timer 120 may also be implemented in software via processing unit 114. For example and without limitation, in some embodiments, controller 112 may be realized via a microcontroller or similar, such as an Arduino™ microcontroller. In addition, some embodiments may further include at least one ambient thermal probe 122, as will be discussed further below.

Generally, controller 112 is operable to, in real-time or at small time intervals via input/output interface 116, receive temperature readings from thermal probe 110 and send corresponding control signals to heater 108 so as to maintain the temperature reading at or above a preset temperature for a corresponding preset time period suitable to disinfect article 104 without inflicting thermal damage thereto. In some embodiments, thermal probe 110 may be operationally linked to controller 112 via a wired or wireless connection, although various connection means may be used, as will be discussed below.

In some embodiments, different sets of disinfection temperature/duration may be pre-determined and stored on controller 112 via internal memory 118. These may be programmatically executed by processing unit 114 therein to control timer 120 and heater 108.

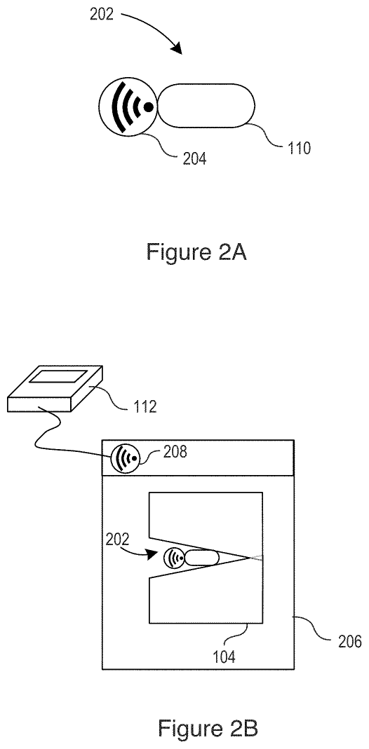

As illustrated in FIGS. 2A and 2B, in some embodiments, thermal probe 110 may be integrated in a wireless probe 202 comprising a wireless transmitter 204 (such as an RF antenna/transmitter or similar) operatively connected to thermal probe 110, so that the probe may be more easily disposed in or on article 104 at strategic locations, such as recess 106 for example, and further optionally while article 104 is sealed in a submersible compartment 206, such as waterproof bag or pouch. This may be done if item 104 may be damaged or degraded if put directly in water. Thus, in some embodiments, input/output interface 116 may be connected or linked to a corresponding RF receiver 208, to receive the temperature reading from wireless probe 202. In some embodiments, RF receiver 208 may be integrated to or disposed on submersible compartment 206 and connected to controller 112 via a wire or cable, to minimize the distance travelled in water of the RF signal. Other embodiments may be considered wherein RF receiver 208 is instead located on receptacle 102, or integrated or embedded within controller 112.

In some embodiments, RF antenna/transmitter 204 may be integrated with thermal sensor 110 on a same miniaturized chip, or again be wired to miniaturized temperature chip, so that the thermal sensor 110 goes in recess 106 while a short-wired RF transmitter 204 substantially extends out of recess 106.

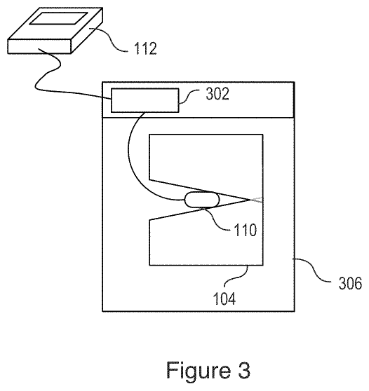

As illustrated in FIG. 3, in some embodiments, temperature probe 110 may be harnessed to a sealed connector or wiring interface 302 integrated or otherwise provided with submersible compartment 306 so to be operatively coupled thereto, with appropriate wired or wireless connector relaying harnessed temperature readings to controller 112.

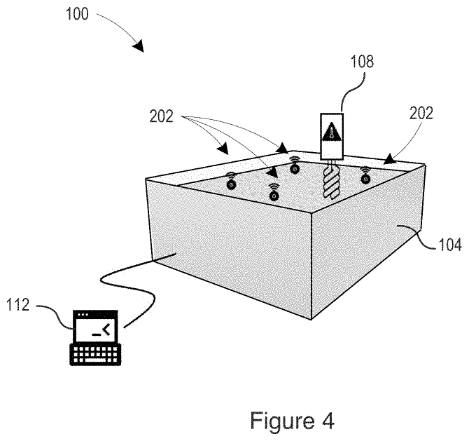

As mentioned above, in some embodiments, one or more ambient thermal probes 122 may also be used to measure ambient temperature around article 104. These may be distributed in receptacle 102, as illustrated in FIG. 4. Controller 112 may take into account temperature readings from ambient probes 122 in addition to the temperature readings from thermal probe 110 so as to better ensure that the temperature does not exceed a preset thermal damage threshold.

In some embodiments, controller 112 may further comprise or be connected to an input device such as a keyboard, touch screen or any other input device known in the art; and to an output device such as a pixel display or similar (for example a built-in display), so as to let a user operate controller 112 and control the thermal disinfection process.

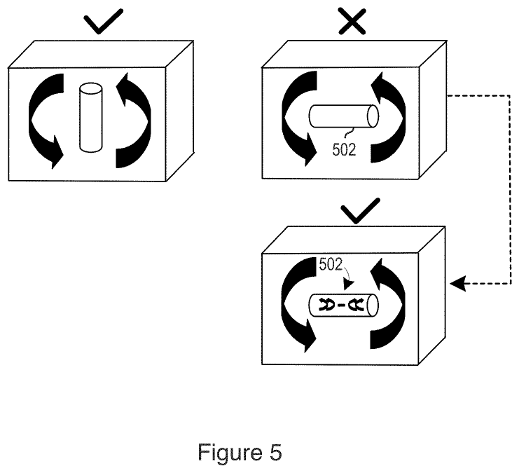

As illustrated in FIG. 5, in some embodiments, device 100 may further comprise a mixing device or unit to generate fluid circulation around article 104 or compartment 206. This may be useful in examples where article 104 is positioned or shaped so as to inhibit fluid circulation. In some embodiments, the mixing device may take the form of a linear magnet 502 operable to controllably rotate under the effect of an induction device (not shown). Rotation of linear magnet 502 thus induces a flow that may be controlled. In other embodiments, the device may include a rotating platform or agitator that can otherwise rotate or agitate the article to be disinfected during a given disinfection cycles so to enhance a uniform heated water distribution.

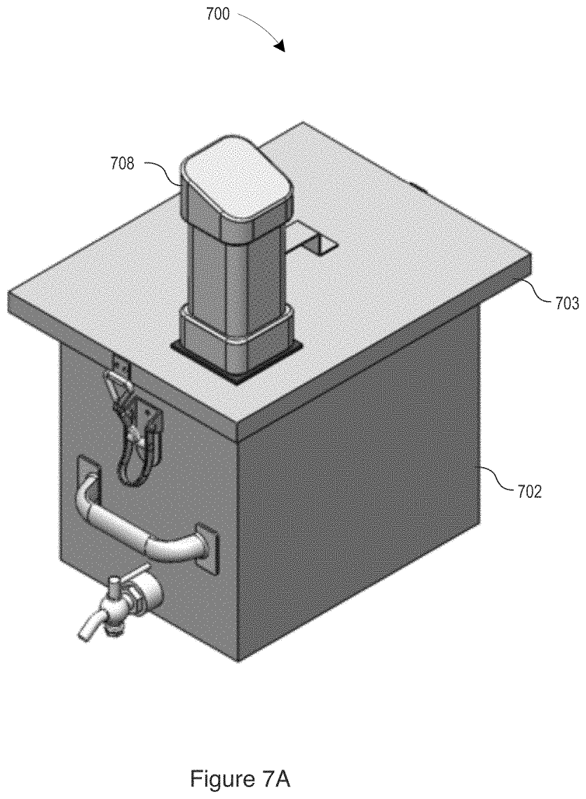

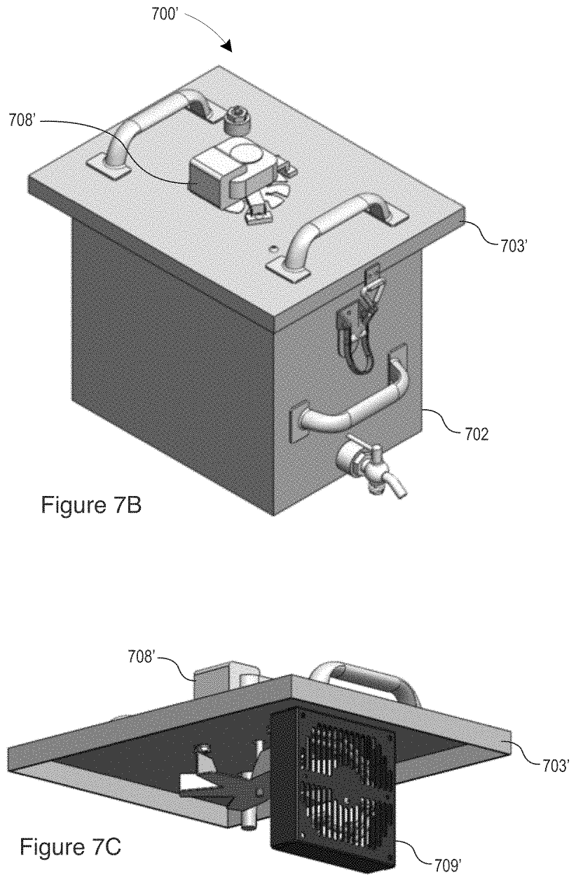

With reference to FIG. 7A, a liquid-based thermal disinfection device 700 will now be described, in accordance with another embodiment. In this embodiment, the device 700 comprises receptacle 702, generally fillable with water or other wet heating liquid and dimensioned to hold a target disinfection article submerged therein. The receptacle is thermally sealable via a lid or like closure 703 that has a liquid heating unit 708 operatively mounted therein. A plurality of thermal probes (not shown) are again provided to be strategically located on or around the target article(s) when the article is submerged to measure a respective water temperature and ultimately control the heating unit so to maintain the temperature reading at or above a preset temperature for a corresponding preset time period suitable to disinfect the article without inflicting damage to the article.

With reference to FIGS. 7B and 7C, a similar thermal disinfection device 700′, this time operable for gas or air-based thermal disinfection, comprises an air heating unit 708′ and collaboratively operable heat distribution device (e.g. heat distribution fan 709′) mounted or otherwise fitted to the thermally sealable lid 703′.

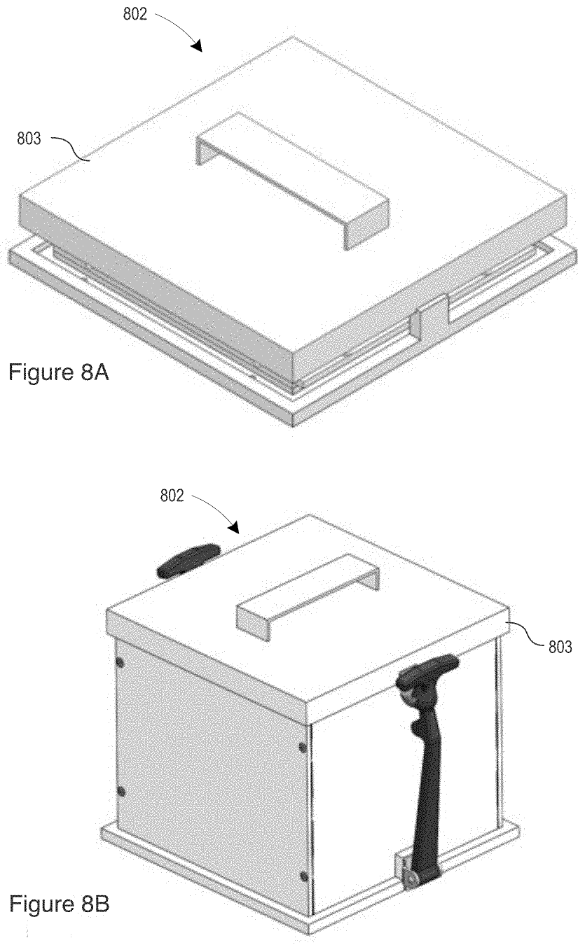

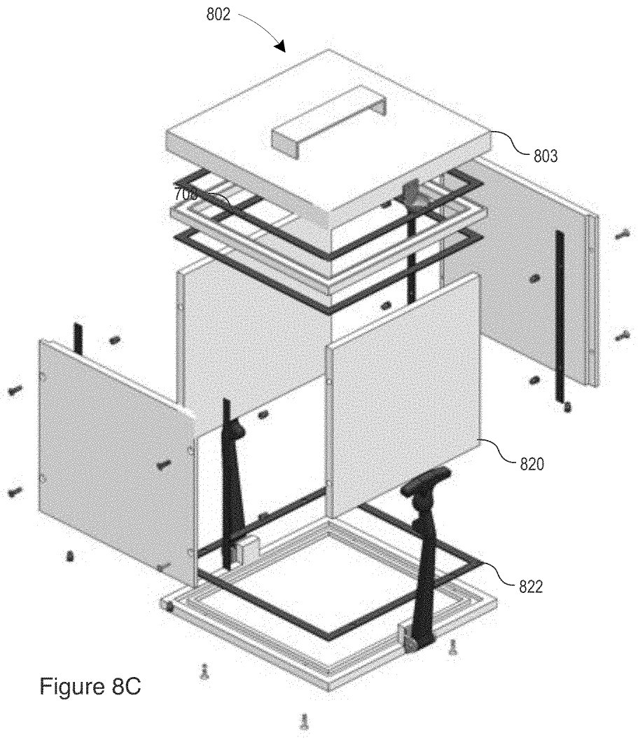

With reference to FIGS. 8A to 8C, a deployable/collapsible receptacle 802 for a thermal disinfection device will now be described, in accordance with another embodiment. In this embodiment, the receptacle 802 can be deployed from a collapsed state, as shown in FIG. 8A, in which all necessary panels and hardware can be stacked and contained for ease of transportation or shipment, to a deployed or fully assembled state, as shown in FIG. 8B. As shown in FIG. 8C, each side panel 820 can be sealably assembled via a corresponding set of gaskets or seals 822, so to provide a thermally sealable receptacle, for either air or water-based heating upon securing the sealable lid 803 thereto. Indeed, in one embodiment, waterproof gaskets can be used between the sheets, with screws, fasteners and/or latches used to securely hold the structure together. Given the deployable/collapsible nature of the design, the device can be shipped and/or transported easily in its collapsed state, and deployed, for example, in the field, in a temporary setting such as a clinic, field hospital or temporary setting, and disassembled post-use to be transported and used elsewhere as needed.

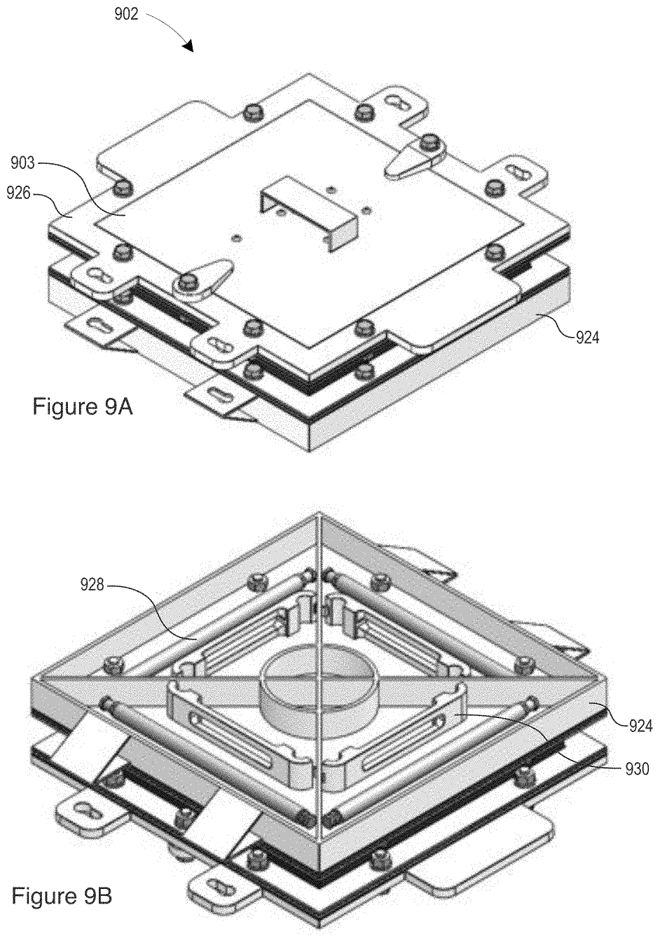

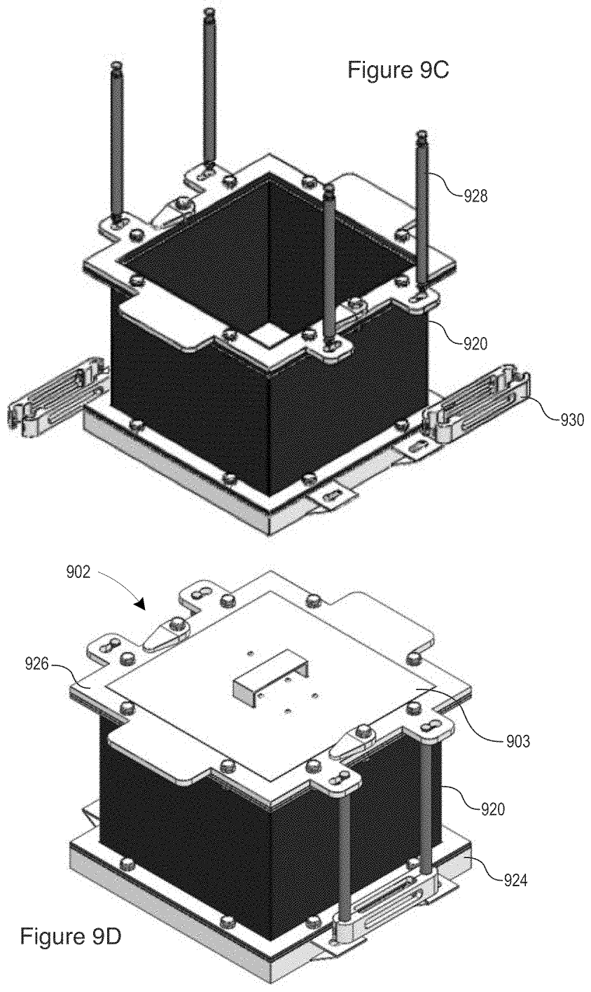

With reference to FIGS. 9A to 9D, a deployable/collapsible receptacle 902 for a thermal disinfection device will now be described, in accordance with another embodiment. In this embodiment, the receptacle 902 can be deployed from a collapsed state, as shown in FIG. 9A, to a deployed or fully assembled state, as shown in FIG. 9D. In its collapsed state, a continuous wraparound or tube-shaped material side panel 920 is conveniently folded in between a structural base 924 and top portion 926, while mountable structural vertical beams (e.g. tie-rods 928) and corresponding securing hardware 930 are conveniently stored within the base 924 (see FIG. 9B) to be mounted and secured as shown in FIG. 9C. Depending on the disinfection medium, different materials may be considered, such as plastics or rubberized materials or fabrics that can provide for a desired level of thermal containment, as well as reduce any gas (air) or liquid (water) exiting the receptacle in use (i.e. air or water impermeable materials). A thermally sealable lid 903 is again provided to enclose a target thermal disinfection article within the receptacle 902 for liquid or gas-based thermal disinfection, as described herein.

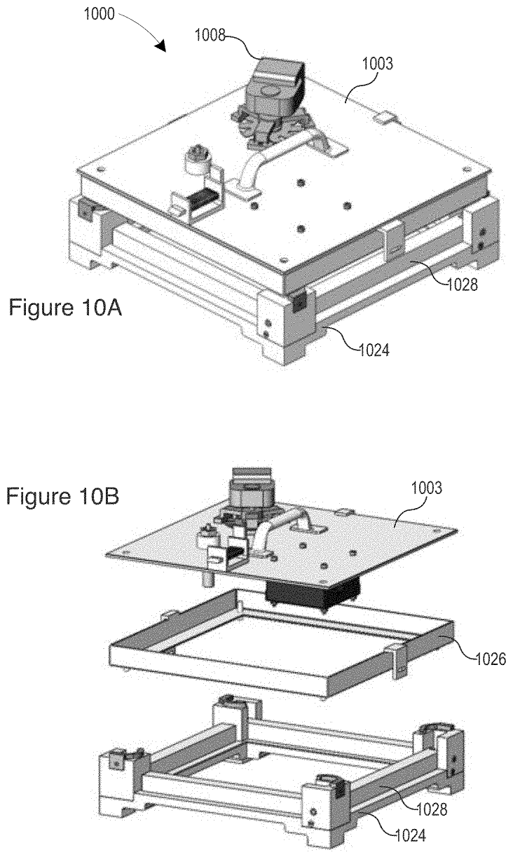

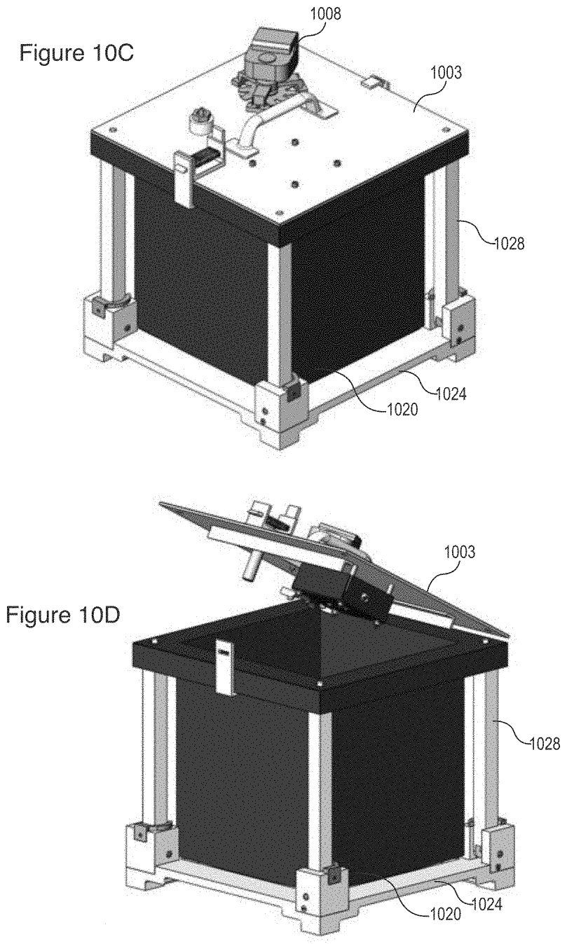

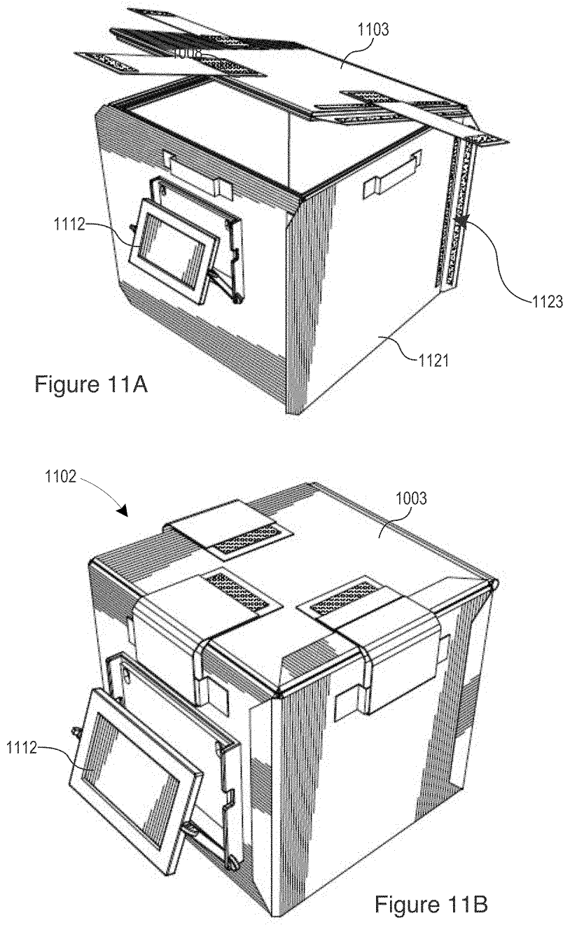

With reference to FIGS. 10A to 10D, a deployable/collapsible thermal disinfection device 1000, comprising a deployable/collapsible receptacle, will now be described, in accordance with another embodiment. In this embodiment, the receptacle can again be deployed from a collapsed state, as shown in FIG. 10A, to a deployed or fully assembled state, as shown closed and opened in FIGS. 10C and 10D, respectively. In its collapsed state, deployable structural vertical beams 1028 are conveniently stored and pivotally deployable from a base 1024 portion to be secured in a vertical position (e.g. via spring-loaded pins) and structurally support a top portion 1026 in forming a frame for the receptacle in its deployed state, in which a water and/or air impermeable fabric bag or sac 1020 can be secured to define the receptacle's thermal disinfection volume. In the illustrated embodiment, an air heating unit 1008 is shown mounted through the device's lid 1003. Again, depending on the disinfection medium, different materials may be considered, such as plastics or rubberized materials or fabrics that can provide for a desired level of thermal containment, as well as gas and/or liquid impermeability.

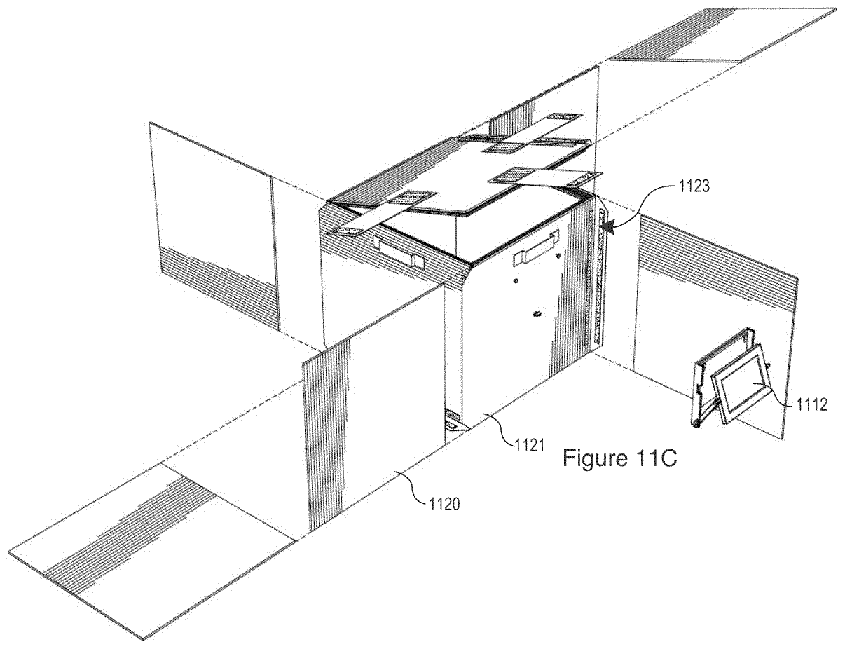

With reference to FIGS. 11A to 11C, a deployable/collapsible thermal disinfection receptacle 1102 will now be described, in accordance with another embodiment. In this embodiment, the receptacle 1102 is deployed by inserting a set of structural bottom, top and side panels 1120 within corresponding panel sleeves 1123 (having hook and loop flap closures) formed within a generally cubic foldable/collapsible fabric shell 1121 manufactured of a gas (air) or liquid (water) impermeable material. A thermal seal gasket or the like 1122 is also fitted within an opening periphery of the shell to form a thermally sealed closure with a flit top lid 1103 of the receptacle (having hook and loop secure closure straps). In this illustrated embodiment, a wireless control panel 1112 can also be secured to the assembled or deployed receptacle 1102 to wirelessly communicated with an internal heating unit and thermal sensor(s) (not shown).

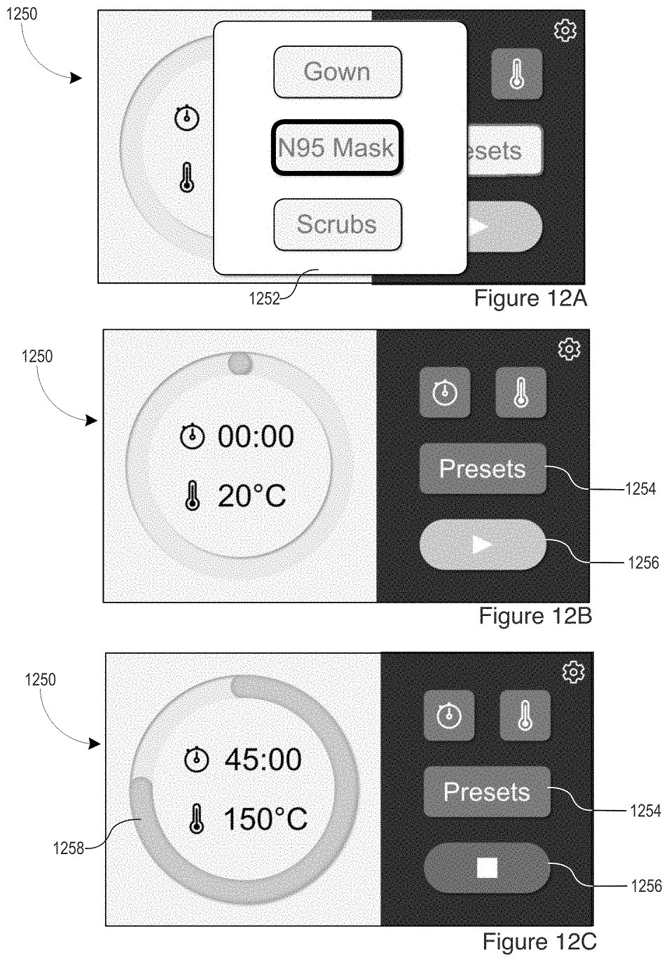

With reference now to FIGS. 12A, 12B and 12C, a digital user interface 1250, for example rendered and operable on a wireless control panel such as panel 1112 of FIG. 11A, will now be described. The digital user interface 1250 generally comprises different operable functions and features to control a thermal disinfection device as described herein. For example, a “Presets” touchscreen button 1254 may be activated to allow the operator to select from a number preset target disinfection articles selectably displayed in a presets window 1252. Each preset may have stored in association therewith a preset time and temperature for a desired or prescribed disinfection level, and may include set instructions for adequate placement or arrangement of thermal probes for consistent and predictable disinfection level monitoring and/or certification. An ON/OFF button 1256 is also provided to start or stop a disinfection cycle. A temperature and elapsed time interface 1258 is also provided to track a progress of a selected cycle. Temperature or timer settings may also, or alternatively be manually adjusted via corresponding menus or functions, as can other operating functions and features be included with the interface, in accordance with different embodiments.

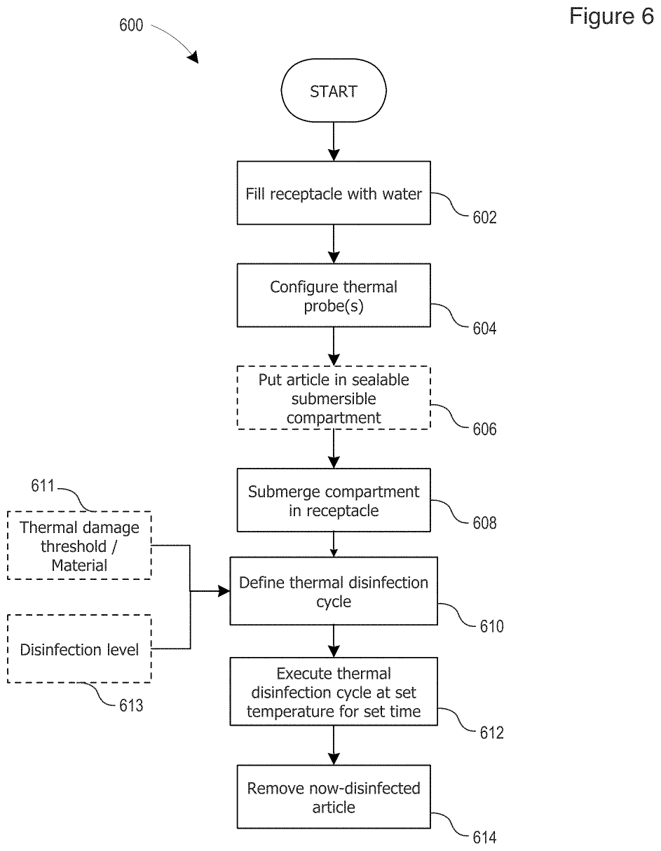

With reference to FIG. 6, and in accordance with one exemplary embodiment, a process for disinfecting an item via system 100, generally referred to using the numeral 600, will now be described. In this exemplary embodiment, process 600 requires little training, which may allow device 100 to be used in a wide range of application settings such as small business and homes. In some embodiments, the designated heating temperature and the duration of the heating process is chosen so as to deliver a corresponding level of disinfection. For example, much of the fabric used for medical personal protection equipment (PPE) can withstand heating above 50° C. for some time, but not excessively more. Indeed, most micro-organisms experience necrosis at about 50° C. With increasing temperature, the death rate increases much faster than the changes caused purely by increased chemical reaction rates. This effect can be used as an advantage to kill micro-organisms faster without doing damage to the items.

Thus, initially, at step 602, if not already done, receptacle 102 is filled with water. Receptacle 102 needs to be filled once and the same water may be reused for multiple disinfection cycles. Naturally, air-based disinfection embodiments will skip this step.

At step 604, thermal probe 110 is configure or placed on article 104, as discussed above. Thus, usually thermal probe 110 would be placed at a hard to reach location, such as recess 106 or other. If the article is manufactured of different materials, probes may be strategically located to monitor effective thermal disinfection temperature of different materials, at different locations, or in different preset or predetermined configurations, In some embodiments, if required, thermal probe 110 may need to be connected via a wire to wiring interface 302, which is connected to controller 112.

Then, at step 606, the article 104 to be disinfected is placed in sealable submersible compartment 206 of the correct size. If no compartment is used, then step 606 may be omitted. Again, this step may be omitted for air-based disinfections.

At step 608, article 104 (or compartment 206) is submerged under water in receptacle 102.

At step 610, the thermal disinfection cycle is defined, for example, based on one or more inputs from the operator such as a preset maximum temperature/thermal damage threshold or item material 611, and/or a level of disinfection required 613, to name a few examples. Other examples may include, but are not limited, preset disinfection cycles with proven, certified or otherwise confirmed disinfection levels for preset or designated articles (e.g. for suggested or prescribed thermal probe configurations).

Generally, device 100 is configured so as to be simple to use, only requiring a short setup and the push of a button. In some embodiments, pre-recorded examples of articles or materials may be provided to the operator. In other embodiments, a distinctive identification number may be provided to device 100 to properly identify article 104, for example a serial number or similar. In other embodiments, device 100 may be operable to automatically identify article 104, via the use of a camera or similar (not shown) and machine vision algorithms.

Once the start button is pressed, at step 612 the process is subsequently automated to ensure the desired or pre-set disinfection level. In some embodiments, the operator may monitor progress using a built-in display. Controller 112 automatically controls heater 108 so to maintain the temperature reading from thermal probe 110 (and if applicable ambient probes 122) at or above a preset temperature for a corresponding preset time period suitable to disinfect the article without inflicting thermal damage to the article.

For example, in some embodiments, a 5 log 10 level of disinfection may be achieved on equipment made of metals such as stainless steel, sensitive polymers such as Teflon™ and rubber o-rings by using a temperature of 70° C. for a duration of about 20 minutes. In other embodiments, a 6 log 10 level of disinfection may be achieved by increasing the temperature incrementally. Different disinfection levels, temperature thresholds, and corresponding disinfection cycle times or periods may be preset or stored in a digital memory or chip, and accessed by the controller based on the operator input, thereby allowing for a certain level of cycle customization for different materials, applications and/or contexts.

Once disinfection is completed, at step 614 the operator may remove the now-disinfected article 104 from compartment 206. In some embodiments, device 100 may be reused quickly, requiring only that the processed items be dried. This may be done passively by allowing the items to sit, or actively with air blow drying to accelerate the process.

In accordance with one exemplary embodiment, a thermal disinfection device is provided that can be operated by a single person with limited to no technical training and meet any one or more of the following criteria: requires limited training or qualification, rugged design for use practically anywhere, mobile and transportable by a single person, versatile and usable for different items to be processed, usable with items not originally designed to be disinfected, certified to be used for personal protective equipment (PPE), compliant to recognized international standards for disinfection, no chemicals or hazardous radiation or any special agent, scalable in size, cost-effective and easy maintenance.

As described above, a given embodiment may be operable to head a fluid such as air or water, prevent leakage of contaminated fluid, reduce heat loss to the environment, facilitate transport (easy to assemble and disassemble, have an internal geometry to facilitate fluid motion and thus increase heat transfer, capable of meeting specification of recognized standards (ISO 15883-6 and AAMI TIR30).

As demonstrated by the examples detailed below, a given embodiment may be operable to achieve a disinfection level of at least 5Log10, operate at a temperature of at least 100C, and optionally provide for safe transportation during operation (e.g. exterior surface and handles to remain below 40C. Electrical components to operate from within the receptacle should be able to withstand operational temperatures (e.g. 100C).

In transportable embodiments, a device may advantageously manufactured to remain within established guidelines for weight (e.g. 23 kg with water for single-user lifting, 46 kg with water for two-user lifting).

Embodiments may also be manufactured to be easy to clean or disinfect, e.g. via self-cleaning through high internal temperature and/or via chemical cleaning using basic agents such as phenolic acids, alcohol (e.g. 70% ethanol), chlorine (e.g. sodium hypochlorite), ammonia solutions (e.g. 10% ammonia).

As outlined below, various tests were conducted to validate the above-described embodiments and designs, notably to sufficiently disinfect items such as N95 masks, surgical masks, face shields, googles and surgical gowns, for example. In this example, tests were conducted on cultures of mycobacterium hassiacum due to high tolerance for high temperatures, and as a substitute for the COVID-19 virus. The cultures were incubated in the test device at various temperatures and times, and a sample of the recuperated area was spread on agar plates and allowed to grow for up to 7 days. The process was completed for various temperatures (45C-85C), times, and materials (N95 mask—mask, nose clip, strap; lab coat/plastic; face shield—shield, plastic support, comfort foam; lab safety glasses).

TABLE 1
Test results on disposable N95 mask

| | | Amount of | |
|---|---|---|---|
| Temp | Time | M. hassiacum | Log10 |
| (° C.) | (min) | (CFU/mL, n = 2) | Redution |
| Starting culture | | 2.80E+08 | — |
| Negative control | | 0.00E+00 | — |
| 85 | 5 | 0.00E+00 | >7 |
| | 15 | 0.00E+00 | >7 |
| | 30 | 0.00E+00 | >7 |
| | 60 | 0.00E+00 | >7 |
| 75 | 5 | 0.00E+00 | >7 |
| | 15 | 0.005+00 | >7 |
| | 30 | 2.50E+03 | 5.02 |
| | 60 | 5.00E+03 | 4.72 |
| 65 | 5 | 7.21E+06 | 1.59 |
| | 15 | 6.62E+06 | 1.63 |
| | 30 | 7.73E+06 | 1.59 |
| | 60 | 5.04E+06 | 1.74 |
| 55 | 5 | 6.90E+06 | 1.61 |
| | 15 | 6.45E+06 | 1.64 |
| | 30 | 6.96E+06 | 1.60 |
| | 60 | 7.04E+06 | 1.60 |

TABLE 2
Test results on various materials for 30 minutes

| | | AIR | | WATER | |
|---|---|---|---|---|---|
| | | Amount of | | Amount of | |
| Temp | Explication | M. hassiacum | Log10 | M. hassiacum | Log10 |
| (° C.) | of Matertal | (CFU/mL) | Reduction | (CFU/mL) | Reduction |
| Starting culture | | 3.70E+06 | — | 7.21E+08 | — |
| Negative control | | 0.00E+00 | — | 0.00E+00 | — |
| 85 | Plastic Lab Coat | 0.00E+00 | >5 | 0.00E+00 | >7 |
| | Face shield- | 0.00E+00 | >5 | 0.00E+00 | >7 |
| | Plastic Shield | | | | |
| | N95 Mask | 0.00E+00 | >5 | 0.00E+00 | >7 |
| | Lab safety glasses | 0.00E+00 | >5 | 0.00E+00 | >7 |
| 80 | Plastic Lab Coat | 4.63E+03 | 2.90 | 0.00E+00 | >7 |
| | Face shield- | 0.00E+00 | >5 | 0.00E+00 | >7 |
| | Plastic Shield | | | | |
| | N95 Mask | 0.00E+00 | >5 | 0.00E+00 | >7 |
| | Lab safety glasses | 0.00E+00 | >5 | 0.00E+00 | >7 |
| 75 | Plastic Lab Coat | 0.00E+00 | >5 | 0.00E+00 | >7 |
| | Face shield- | 0.00E+00 | >5 | 0.00E+00 | >7 |
| | Plastic Shield | | | | |
| | N95 Mask | 0.00E+00 | >5 | 0.00E+00 | >7 |
| | Lab safety glasses | 0.00E+00 | >5 | 0.00E+00 | >7 |

TABLE 3
Test results on various materials for 30 minutes

| | | AIR | | WATER | |
|---|---|---|---|---|---|
| | | Amount of | | Amount of | |
| Temp | Explication | M. hassiacum | Log10 | M. hassiacum | Log10 |
| (° C.) | of Matertal | (CFU/mL) | Reduction | (CFU/mL) | Reduction |
| Starting culture | | 5.22E+08 | — | 1.30E+08 | — |
| Negative control | | 0.00E+00 | — | 0.00E+00 | — |
| 85 | N95 Mask-Strap | 1.97E+05 | 3.42 | 0.00E+00 | >7 |
| | N95 Mask- | 0.00E+00 | >7 | 0.00E+00 | >7 |
| | Nose clip | | | | |
| | N95 Mask- | 0.00E+00 | >7 | — | — |
| | Nose foam | | | | |
| | Face shield- | 1.42E+05 | 3.57 | 0.00E+00 | >7 |
| | Plastic Support | | | | |
| 75 | N95 Mask-Strap | 0.00E+00 | >7 | 0.00E+00 | >7 |
| | N95 Mask- | 0.00E+00 | >7 | 0.00E+00 | >7 |
| | Nose clip | | | | |
| | N95 Mask- | 0.00E+00 | >7 | 0.00E+00 | >7 |
| | Nose foam | | | | |
| | Face shield- | 0.00E+00 | >7 | — | — |
| | Plastic Support | | | | |

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A thermal disinfection device for disinfecting an article susceptible to thermal damage, the device comprising:
a receptacle dimensioned to hold the article within a heating fluid;
a heater;
a plurality of thermal probes dimensioned and independently displaceable to be located at various locations on, in or within the article when in said receptacle, to measure a respective temperature of the heating fluid at each of said locations; and
a controller operable to receive as input respective temperature readings from said plurality of thermal probes from said various locations, and control said heater so to maintain said temperature reading at or above a preset temperature for a corresponding preset time period suitable to disinfect the article without inflicting damage to the article.

2. The device of claim 1, wherein each of said plurality of thermal probes comprises a wireless thermal probe.

3. The device of claim 2, wherein the device further comprises a wireless receiver operatively coupled to the controller for receiving said respective temperature readings.

4. The device of claim 2, wherein the heating fluid comprises water, and wherein the device further comprises a sealable submersible compartment in which to dispose the article, wherein said compartment comprises a wireless receiver disposed thereon to wirelessly interface with each said wireless thermal probe from within said compartment.

5. The device of claim 1, wherein the heating fluid comprises water, and wherein the device further comprises a sealable submersible compartment in which to dispose the article, wherein said compartment comprises a wiring interface to wired connections with each of said plurality of thermal probes from within said compartment, and an external wired or wireless connection via said wiring interface to said controller.

6. The device of claim 1, further comprising an ambient thermal probe to measure an ambient temperature around the article, wherein said controller is further operable to limit said ambient temperature exceeding a preset thermal damage threshold.

7. The device of claim 1, further comprising a mixing device for enhancing fluid circulation within said receptacle.

8. The device of claim 7, wherein the fluid comprises water and wherein said mixing device comprises an induction-controlled rotating linear magnet.

9. The device of claim 7, wherein the fluid comprises air and wherein said mixing device comprises a fan.

10. The device of claim 1, wherein said preset temperature is about 70 C and said preset time period is about 20 minutes.

11. The device of claim 1, wherein the controller further comprises an interface to adjust said preset temperature, and wherein said controller is operable to automatically adjust said corresponding preset time period according to said adjusted preset temperature.

12. The device of claim 1, wherein the controller further comprises a user interface providing for selection from a plurality of preset disinfection cycles preset for a corresponding set of designated articles having a preset disinfection temperature and time associated therewith consistent with a previously tested disinfection level.

13. The device of claim 1, wherein said plurality of thermal probes are dimensioned so to be disposable within a recess or cavity of the article.

14. The device of claim 1, wherein said receptacle is hermetically sealed in operation for air-based thermal disinfection.

15. The device of claim 1, wherein at least some of said receptacle is manufactured of a collapsible material to allow for collapse and deployment of the device.

16. The device of claim 15, wherein said collapsible material comprise a water impermeable fabric.

17. The device of claim 1, wherein the heating fluid is selectable between water or air.

18. A method for disinfecting an article susceptible to thermal damage, the method comprising:
locating a plurality of thermal probes on, in or within the article in accordance with a preset probe layout defined for the article;
containing the article within a heating fluid;
heating the heating fluid so to maintain respective temperature readings from said plurality of thermal probes at or above a preset disinfection temperature for a corresponding preset time period suitable to disinfect the article without inflicting thermal damage to the article.

19. The method of claim 18, further comprising, prior to said heating, selecting a preset disinfection cycle from a plurality of selectable preset disinfection cycles defined for different articles and/or disinfection levels, wherein said heating is automatically implemented as a function of said selecting.

20. A thermal disinfection device for disinfecting an article having multiple recesses defined therein and susceptible to thermal damage, the device comprising:
a receptacle dimensioned to hold the article submerged in water;
a heater;
a plurality of thermal probes dimensioned and independently displaceable to be located within the recesses when the article is submerged in water in said receptacle, to measure a respective water temperature within each of the recesses; and
a controller operable to receive as input respective temperature readings from said plurality of thermal probes from within the recesses, and control said heater so to maintain said temperature reading at or above a preset temperature for a corresponding preset time period suitable to disinfect the article without inflicting damage to the article.
